(19) United States
(12) Patent Application Publication
(10) Pub. No.: US 2008/0300522 A1
(43) Pub. Date: Dec. 4, 2008

(54) Abdominal belt

(76) Inventors: Charlene Chen, San Francisco, CA (US); Nam C. Quach, San Francisco, CA (US)

Correspondence Address: DERGOSITS & NOAH LLP, FOUR EMBARCADERO CENTER, SUITE 1450, SAN FRANCISCO, CA, 94111, US

(21) Appl. No.: 11/809,516

(22) Filed: Jun. 1, 2007

(51) Int. Cl. A61F 5/00; A61F 5/02; A61F 5/28

(52) U.S. Cl. 602/19; 128/100.1; 2/44

(57) ABSTRACT

A device for supporting the abdominal region of a user. A rectangularly-shaped stretch material having first and second distal ends and fixture elements proximate said distal ends are provided for facilitating the releaseable joinder of them about the abdominal region of a user. A plurality of straps are affixed at the mid-point of the stretch material which, in turn, support rings. A series of stretchable bands pass through the rings and in turn are each connected to a pair of connectors such that when the connectors are joined, the stretchable bands provide a force against the stretch material urging the distal ends toward the mid-point of this material.

### TECHNICAL FIELD

The present invention involves a device for supporting the abdominal region of a user. The device is particularly advantageous in restoring abdominal muscle and control particularly after pregnancy, an event which generally results in the stretching of abdominal muscularature and resultant extension.

### BACKGROUND OF THE INVENTION

There are a number of devices intended to encircle the waist and lower back of a user for a plethora of purposes. Some of these devices act as back supports for many people suffer from lower back pain as a result of scoliosis or weak back muscles which do not adequately support the lower spine. Other products, including girdles and undergarment foundations are employed for aesthetic reasons causing a slimming effect under a dress or pants. Yet further articles of this nature are offered as medical appliances for such widely divergent uses as hernia support apparatus and bandage retention mostly employed post surgically.

Virtually all of the appliances discussed above commonly employ a single and, in some instances, multiple layers of stretch fabric which cinches upon a user causing abdominal and lower back compression. Even when multiple stretchable fabric layers are employed, they are invariably used as nothing more than redundant co-fabric with the notion being that if a single layer of stretchable fabric is good, then multiple layers of the same fabric must be better. There are, however, certain inherent disadvantages in taking such an approach.

When multi-layer stretchable foundation garments are employed, a user often experiences constriction resulting in limited movement. This is particularly the case when such multi-layer stretchable garments are applied to the lower back of a user which caused undue restriction in bending and twisting motions.

In addition to the above, it has been determined that few if any stretchable foundation garments have been designed to assist in a woman's attempt to rebuild abdominal muscles and consequently reestablish a relative flat stomach and pre-pregnancy conformity after the birth of a child. Pregnancy is unique in its causation of stretch and oftentimes ruptured abdominal musculature, a state which many woman never recover from. It is common place for a woman to reminisce about the shape she had before pregnancy resigning herself to the realization that her pre-pregnancy look would never be restored. Virtually all elasticized or stretchable foundation garments currently known are incapable of assisting in the rebuilding of abdominal musculature.

It is thus an object of the present invention to provide a multi-layered stretchable garment which is capable of providing abdominal support while overcoming the disadvantages recited above.

It is yet a further object of the present invention to provide a foundation garment which is able to restore a woman's pre-pregnancy shape after childbirth.

These and further advantages will be more readily apparent when considering the following disclosure and appended claims.

### SUMMARY OF THE INVENTION

The present invention is directed to a device for supporting the abdominal region of a user comprising a substantially rectangularly-shaped stretch material having first and second distal ends and fixture elements proximate said distal ends for facilitating the releaseable joinder of said distal ends and stretching of said stretch material upon the encirclement of said stretch material about the lower back and abdomen of said user. A plurality of straps are affixed substantially at the mid point of said stretch material each of said straps supporting ring at each of its extreme ends. A pair of connectors are provided, each being appended to a first end of a series of stretchable bands, each of said stretchable bands being received by one of said rings and each terminating proximate said distal ends of said substantially rectangularly-shaped stretchable material such that as said connectors are releaseably joined proximate the distal ends of said stretch material, said stretchable bands are caused to pull again said distal ends and urge them toward said mid-point of said stretchable material.

### BRIEF DESCRIPTION OF THE FIGURES

FIG. 1 is a perspective view of the device of the present invention.

FIG. 2 is the device of FIG. 1 showing the effect of applying a stretching force to a portion of said device and the reaction that such a force has upon the device in use.

FIG. 3 is a perspective view of a user of the present invention depicting the application of the device in operation.

FIG. 4 is a perspective view of a user of the present invention depicting a preferred embodiment thereof.

### DETAILED DESCRIPTION OF THE INVENTION

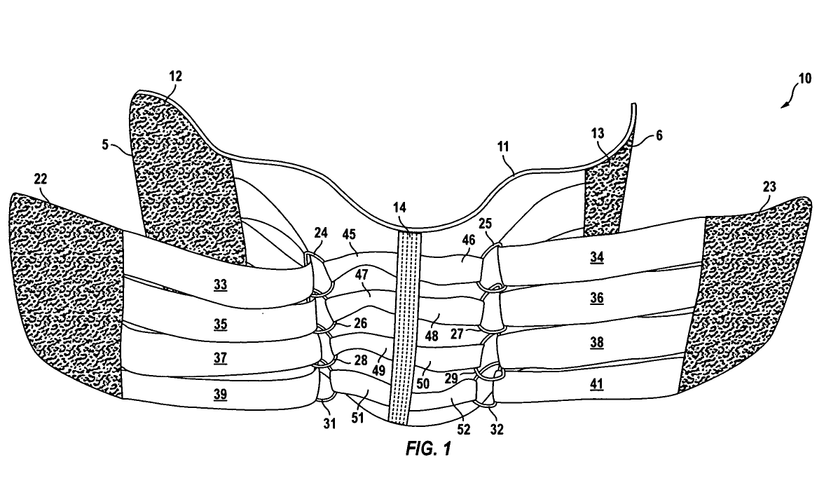

Turning first to FIG. 1, device 10 is depicted for supporting the abdominal region of a user, its position being shown in FIG. 3 as device 10 resides properly upon user 30.

Device 10 comprises substantially rectangularly-shaped stretch material 11 comprised of, for example, neoprene or other lightweight stretchable fabric capable of returning to its original dimension repeatedly upon multiple use. Rectangularly-shaped stretch material 11 is provided with first and second distal ends 5 and 6 having fixture elements 12 and 13 proximate thereto facilitating the releaseable joinder of said distal ends and the stretching of said stretch material upon the encirclement of said stretch material about the lower back and abdomen of said user (FIG. 3). Ideally, these fixture elements can comprise hook and loop fasteners such as Velcro® although other fasteners and snaps can be employed for this purpose. A plurality of straps 45 to 52 generally composed of nonstretchable fabric straps are affixed substantially at the mid point of stretch material 11, depicted as being sewn at said mid point along stitch line 14.

Each of straps 45-52 support rings 24 through 29, 31 and 32 at their extreme ends. A series of stretchable bands 33 to 39 and 41 are permanently affixed to a pair of connectors 22 and 23, the stretchable bands being received by said rings 24 through 29, 31 and 32 and each terminating proximate distal ends 5 and 6 by being permanently affixed to fixture elements 12 and 13. In completing this description, it is noted that connector pair 22 and 23 is intended, in use, to reside over fixture elements 12 and 13 thus releaseably securing connector pair 22 and 23 about the abdomen of a user. This connector pair can also be composed of hook and loop fasteners (Velcro@) or any other suitable well known snap or fixture device.

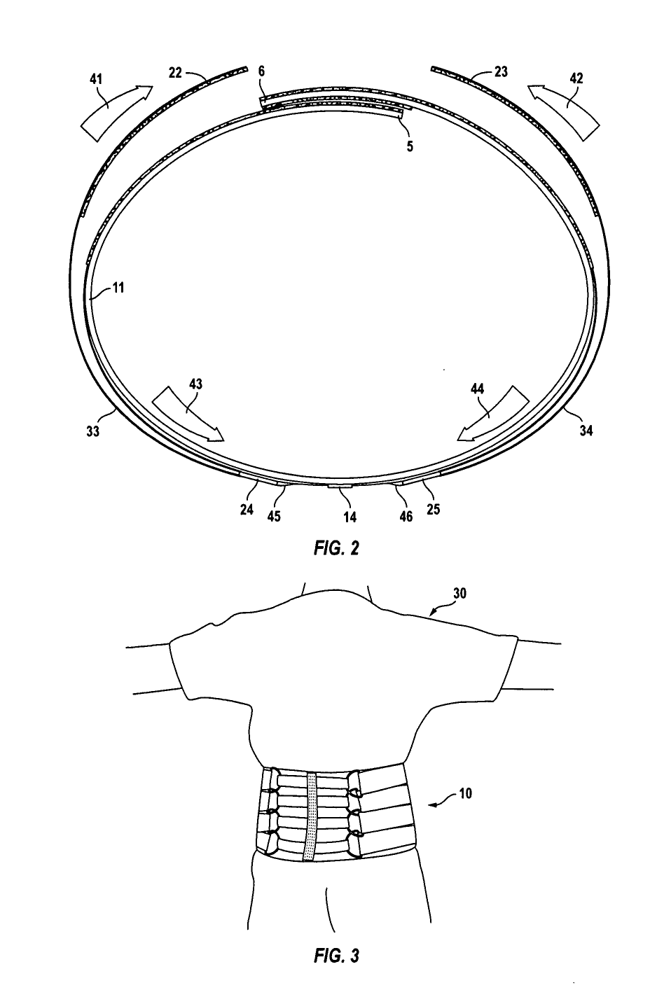

The configuration of the present device as described above with respect to FIG. 1 creates a unique dynamic more readily visualized by referring to FIG. 2. Specifically, it is noted that as connectors 22 and 23 are moved away from centrally located stitch seam 14 in the direction of arrows 41 and 42, stretchable bands 33 through 39 and 41, in passing through rings 24 through 29, 31 and 32 pull against distal ends 5 and 6 as they are fixedly connected to fixture elements 12 and 13 causing rectangularly-shaped stretch material 11 to be urged toward mid-point seam 14 as noted by vector arrows 43 and 44. Thus, as connector pair 22 and 23 are drawn about a user's lower back and abdomen and connect in the abdominal region of a user, the combination of straps 45-52, rings 24 through 29, 31 and 32 and stretchable bands 33 through 39 and 41 cause rectangularly-shaped stretch material 11 to contract towards vertically extending stitch seam 14 applying further compression to the abdominal region of a user. Such dynamic forces are unachievable by the prior art and represent a dramatic functional improvement in operation as compared thereto.

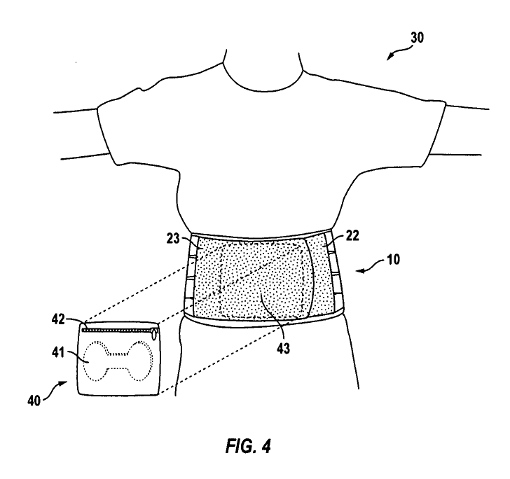

Reference is next made to FIG. 4 depicting a preferred embodiment of the present invention. In this regard, it has been recognized that if a weight was to be placed on a user's abdomen, the benefits of the present invention could be enhanced. Pouch 40 is shown having zippered or other type of closure opening 42 capturing weight 41. Obviously, the size of weight 41 could be varied depending upon a user's needs wherein closure 42 of pouch 40 can simply be selectively opened with weights of varying sizes being positioned therein such as weight 41 as depicted.

Belt 10 can be employed as described previously. When embracing the embodiment of FIG. 4, pouch 40 can be provided with a releaseable attachment means on its surface opposite that can be visualized in FIG. 4. For example, hook and loop fasteners 22 and 23 can engage a complimentary hook and loop surface on the back side of pouch 40 to enable the pouch to reside within field 43. As such, when user 30 is wearing the present device, for example, while in a prone position, pouch 40 containing weight 41 can be selectively appended to area 43 to cause additional pressure to be exerted upon the abdomen of user 30. Again, this can assist in abdominal support and abdominal musculature enhancement.

Yet a further advantage recognized by the present device results from the use of the strap and band combination described above as compared to a continuous piece of stretchable fabric such as that employed by prior art designs. In employing strap and band combinations, a user is able to bend and turn more easily for one is not confronted with a multi-layer set of stretchable fabric at one's lower back region in the employment of the present invention. The present straps and bands move separately from one another and enable a user to engage in virtually a full range of motion with minimal restriction as compared to competitive devices of the prior art. As such, one not only achieves a superior sense of abdominal muscle support, essential in the rebuilding process of a post pregnant user but does so with minimal lower back constriction.

What is claimed is:

1. A device for supporting the abdominal region of a user comprising a substantially rectangularly-shaped stretch material having first and second distal ends and fixture elements proximate said distal ends for facilitating the releaseable joinder of said distal ends and the stretching of said stretch material upon the encirclement of said stretch material about the lower back and abdomen of said user, a plurality of straps affixed substantially at the mid-point of said stretch material each of said straps supporting ring at its extreme end, a pair of connectors, each being appended to a first end of a series of stretchable bands, each of said stretchable bands being received by one of said rings and each terminating proximate said distal ends of said substantially rectangularly-shaped stretchable material such that as said connectors are releaseably joined proximate the distal ends of said stretch material, said stretchable bands are caused to pull against said distal ends and-urge them toward said mid point of said stretchable material.

2. The device of claim 1 wherein said substantially rectangularly-shaped stretch material, and straps and stretchable bands are sized such that upon use, said distal ends of said rectangularly shaped stretch material are releaseably joined over the abdomen of a user and said pair of connectors are releaseably joined over said distal ends.

3. The device of claim 1 wherein said fixture elements and said connectors comprise hook and loop fasteners.

4. The device of claim 1 wherein said substantially rectangularly shaped stretch material comprises neoprene.

5. The device of claim 1 further comprising a pouch removably appended to said pair of connectors, said pouch designed to receive a weight therein.

6. The device of claim 5 wherein said pouch is selectively appended to said pair of connectors through the use of hook and loop fasteners.

7. The device of claim 5 wherein the interior of said pouch is selectively accessible to enable a user to vary the weights contained therein.
